(19) United States
(12) Patent Application Publication
(10) Pub. No.: US 2002/0099297 A1
(43) Pub. Date: Jul. 25, 2002

(54) Inflatable cuff used for blood pressure measurement

(75) Inventors: Tsuneo Nakagawa, Komaki-shi (JP); Takeshi Kurosaki, Komaki-shi (JP)

Correspondence Address: OLIFF & BERRIDGE, PLC, P.O. BOX 19928, ALEXANDRIA, VA, 22320, US

(73) Assignee: Colin Corporation

(21) Appl. No.: 09/768,267

(22) Filed: Jan. 25, 2001

(51) Int. Cl. A61B 5/02

(52) U.S. Cl. 600/494; 600/499; 600/493; 600/496; 606/202

(57) ABSTRACT

An inflatable cuff for being wound and inflated around a body portion pressing a body portion of a living subject to press the body portion in measuring a blood pressure of the subject, the cuff comprising: an inflatable bag which is located in a portion of the cuff in a lengthwise direction thereof and a connector which is attached to a portion of the inflatable bag that is distant, by a predetermined distance, from a distal end of the cuff in a widthwise direction thereof, and which is adapted to connect the inflatable bag to a gas-supply pipe to supply a pressurized gas from the pipe to the inflatable bag.

This application is based on Japanese Patent Application No. 10-220158 filed Aug. 4, 1998, the contents of which are incorporated hereinto by reference.

### BACKGROUND OF THE INVENTION

1. Field of the Invention

The present invention relates in general to an inflatable cuff which is cylindrically wound around a body portion of a living subject so as to press the body portion in measuring a blood pressure of the subject.

2. Discussion of Related Art

There is known an inflatable cuff which is cylindrically wound around a body portion of a living subject so as to press the body portion in measuring a blood pressure of the subject. The inflatable cuff includes a belt-like elongate bag which is formed of a nylon resin cloth, and a rubber-made inflatable bag accommodated in the belt-like elongate bag. Alternatively, the inflatable cuff includes a belt-like elongate bag which is formed by folding a vinyl resin sheet, and a portion of the elongate bag is high-frequency welded so as to define an air-tight inner space functioning as an inflatable bag. With the cuff being wound around a body portion of the living subject, e.g., an upper arm of the subject, a pressurized air is supplied to the inflatable bag of the cuff through an air pipe connected thereto, so that the inflatable bag is inflated to press the body portion. The air pipe is formed of a material which can be elastically deformable, and is connected to the inflatable bag at a distal end of the cuff, which distal end is more distant from the heart of the subject than an opposite proximal end of the cuff, such that the air pipe extends in a direction parallel to the widthwise direction of the cuff.

More specifically described by referring to FIGS. 7 and 8 which respectively show an outer surface and an inner surface of a conventional cuff generally indicated at 80, the cuff 80 has a belt-like elongate bag 82 formed by an outer sheet member 84 and an inner sheet member 86, and an inflatable bag 88 accommodated in a portion of the elongate bag 82 in its lengthwise direction. A pair of fastener pads 90, 92 are respectively provided on the outer sheet member 84 at one of two lengthwise opposite end portions of the cuff 80 which is on the side of the inflatable bag 88, and on the inner sheet member 86 at the other of the two lengthwise opposite end portions of the cuff 80 which is remote from the inflatable bag 88. The fastener pads 90, 92 respectively have a multiplicity of looped fibers, and a multiplicity of elastically deformable hooks, which are disengageably or detachably held in engagement with each other, for thereby permitting the cuff which is wound around the body portion of the subject to be securely fixed relative to the body portion. An air pipe 94 formed of a rubber material is connected to a portion of the inflatable bag 88 which corresponds to a longitudinally intermediate portion of the cuff 80 on the side of its distal end, such that the air pipe 94 extends in a direction parallel to the widthwise direction of the cuff 80.

When it is necessary to continuously monitor the medical condition of the patient, the blood pressure measurement is continuously effected at a predetermined time interval while the inflatable cuff which is connected to the BP measuring apparatus is kept wound around the body portion of the patient. If the conventional cuff 80 constructed as described above is wound around the body portion, e.g., an upper arm 96, the air pipe 94 extending from the distal end of the cuff 80 in the direction parallel to the widthwise direction of the cuff 80 abuts on the inner side of the forearm 64 of the patient, and is bent along the elbow arthrosis of the patient, as shown in FIG. 9. In this case, the air is unlikely to flow through the air pipe 94, and the volume of the inflatable bag 88 is not sufficiently reduced even in a non-BP measurement period during which the inflatable bag 88 is deflated for releasing the body portion of the patient from the pressing force applied from the inflatable bag 88. Accordingly, the skin of the patient is kept in close contact with the inner surface of the cuff even during the non-BP measurement period, and becomes sweaty and sodden, and generate odor. In particular, the patient who is confined to bed and cannot deal with such an uncomfortable condition tends to suffer from the problem.

### SUMMARY OF THE INVENTION

It is therefore an object of the present invention to provide an inflatable cuff used for measuring a blood pressure of a living subject, which cuff prevents an air pipe connected to an inflatable bag from being bent when the cuff is wound around a body portion of the subject, for thereby preventing the skin of the body portion of the subject from becoming sweaty and sodden due to an insufficient air flow through the air pipe experienced when the air pipe is bent.

The above-indicated object of the present invention may be achieved according to a principle of the present invention, which provides an inflatable cuff for being wound and inflated around a body portion of a living subject to press the body portion in measuring a blood pressure of the subject, the cuff comprising: an inflatable bag which is located in a portion of the cuff in a lengthwise direction thereof; and a connector which is attached to a portion of the inflatable bag that is distant, by a predetermined distance, from a distal end of the cuff in a widthwise direction thereof, and which is adapted to connect the inflatable bag to a gas-supply pipe to supply a pressurized gas from the pipe to the inflatable bag.

In the present cuff wherein the connector, to which the gas-supply pipe is connected for supplying a pressurized gas to the inflatable bag, is attached to a portion of the inflatable bag that is distant, by a predetermined distance, from the distal end of the cuff in the widthwise direction thereof, the pipe which is connected to the connector is not bent even when the cuff is wound around the body portion of the subject with the pipe contacting the inner side of the forearm of the living subject, since the connector is distant from the inner side of the forearm by a distance enough to permit the pipe to extend in the direction parallel to the widthwise direction of the cuff without being bent. Accordingly, the present arrangement is effective to prevent the skin of the subject from becoming sweaty and sodden due to an insufficient air flow through the pipe experienced when the pipe is bent without extending in the widthwise direction of the cuff.

In one preferred form of the present invention, the portion of the inflatable bag to which the connector is attached is located, in the lengthwise direction of the cuff, at a position where the connector is externally exposed in a state in which the cuff is wound around the body portion of the subject with two lengthwise opposite end portions of the cuff being superposed on each other.

According to this arrangement, the pipe is not covered with one of the two lengthwise opposite end portions of the cuff which is remote from the inflatable bag when the cuff is cylindrically wound around the body portion of the subject with the above-indicated one of the two lengthwise opposite end portions being superposed on the other of the two lengthwise opposite end portions of the cuff. Accordingly, the pipe is likely to be freely deformed, for thereby preventing the skin of the subject from becoming sweaty and sodden due to an insufficient flow of the gas through the pipe experienced when the pipe is bent without extending in the widthwise direction of the cuff.

In another preferred form of the present invention, the connector comprises a conduit which is adapted to connect the inflatable bag to the gas-supply pipe and which outwardly projects from the inflatable bag such that the conduit is inclined with respect to the inflatable bag.

In this arrangement, the pipe is connected to the connector via the conduit such that the pipe is inclined with respect to the inflatable bag, for thereby preventing the air pipe from being bent.

In still another preferred form of the present invention, the conduit is inclined with respect to the inflatable bag by an angle which falls in a range of from 20 degrees to 40 degrees with respect to the widthwise direction of the cuff in a plane which is perpendicular to a plane of the cuff and is parallel to the widthwise direction thereof.

According to this arrangement, the pipe is connected to the connector via the conduit such that the pipe is inclined with respect to the inflatable bag by an angle which falls in a range of from 20 degrees to 40 degrees with respect to the widthwise direction of the cuff, for thereby preventing the pipe from being bent.

In a further preferred form of the present invention, the connector is formed of an elastic material and is elastically deformable.

Even when the cuff constructed according to this arrangement is wound around a thin upper arm with the connector being covered with one of the two lenthwise opposite end portions of the cuff which is remote from the inflatable bag, the connector is elastically deformed, facilitating winding of the cuff around the upper arm, and permitting the pipe to extend in a direction parallel to the widthwise direction of the cuff, so that gas is permitted to easily flow through the pipe.

The above-indicated object of the present invention may be achieved according to another aspect of the invention, which provides an automatic blood pressure measuring apparatus, comprising: an inflatable cuff according to claim 1; a pressing-force changing device which changes a pressing force of the cuff applied to the body portion of the subject; a determining device which determines a blood pressure of the subject based on a pulse wave produced in the cuff in synchronism with heartbeat of the subject while the pressing force of the cuff is changed by the pressing-force changing device; a negative-pressure producing device which produces a negative pressure lower than an atmospheric pressure; and a deflating device which communicates, after the determining means determines the blood pressure of the subject, the negative-pressure producing device with the inflatable bag of the cuff so that the negative-pressure producing device sucks the gas from the inflatable bag and thereby forcibly deflates the inflatable bag.

According to this arrangement, the cuff deflating device communicates the negative-pressure producing device with the inflatable bag of the cuff after the blood pressure of the subject has been determined, so that the negative-pressure producing device sucks the gas from the inflatable bag and thereby forcibly deflates the inflatable bag. Accordingly, the cuff is loosened around the body portion of the subject, so that the area of contact of the cuff with the skin of the body portion is minimized. Consequently, the amount of gas permitted to flow through the space between the cuff and the subject's skin is greatly increased, so that the skin is effectively prevented from becoming sweaty and sodden.

### BRIEF DESCRIPTION OF THE DRAWINGS

The above and other objects, features, advantages and technical and industrial significance of the present invention will be better understood by reading the following detailed description of a presently preferred embodiment of the invention, when considered in connection with the accompanying drawings, in which:

FIG. 1 is a diagrammatic view of a blood pressure measuring apparatus which includes an inflatable cuff constructed according to one embodiment of the present invention;

FIG. 2 is a plan view of an outer surface of the cuff of FIG. 1;

FIG. 3 is a plan view of an inner surface of the cuff of FIG. 1;

FIG. 4 is a transverse-cross sectional view of the cuff of FIG. 1 taken along line 4-4 of FIG. 2;

FIG. 5 is a view showing the cuff shown in FIGS. 2-4 which is wound around a body portion of a living subject;

FIG. 6 is a flow chart illustrating a control program according to which the apparatus of FIG. 1 operates;

FIG. 7 is a plan view corresponding to FIG. 2, the view showing an outer surface of a conventional cuff;

FIG. 8 is a plan view corresponding to FIG. 3, the view showing an inner surface of a conventional cuff; and

FIG. 9 is a view showing the conventional cuff of FIGS. 7 and 8 which is wound around a body portion of a living subject.

### DETAILED DESCRIPTION OF PREFERRED EMBODIMENT

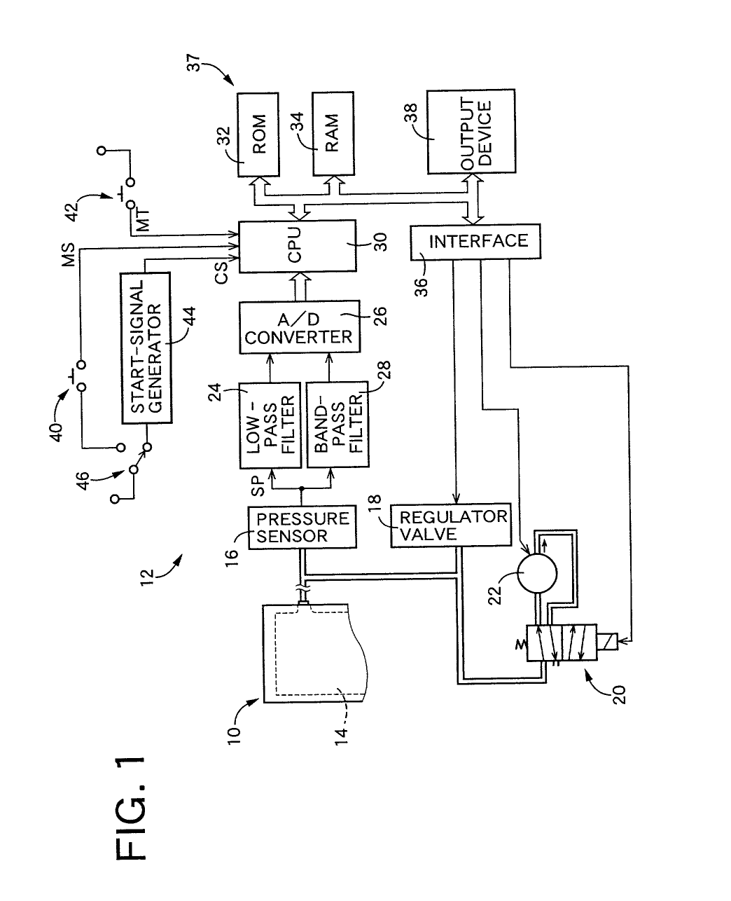

Referring first to FIG. 1, there is schematically shown a blood pressure (BP) measuring apparatus 12 which includes an inflatable cuff 10 constructed according to one embodiment of the present invention. The inflatable cuff 10 includes an inflatable bag 14 connected to a pressure sensor 16 and a pressure regulator valve 18, and an air pump 22 via a selector valve 20. The pressure sensor 16 detects an air pressure in the inflatable bag 14, and generates a pressure signal SP indicative of the detected pressure. The regulator valve 18 functions as a pressing-force changing device which changes a pressing force of the cuff applied to the body portion of the subject. The regulator valve 18 is adapted to slowly or quickly deflate the inflatable bag 14 by changing a cross sectional area of an air passage thereof. The selector valve 20 is a solenoid-operated valve, and is selectively placed in an air-supplying position and an air-discharging position. In the air-supplying position, the selector valve 20 permits the inflatable bag 14 of the cuff 10 to communicate with an outlet of the air pump 22 and permits an inlet of the air pump 22 to communicate with the atmosphere. In the air-discharging position, the control valve 20 permits the inflatable bag 14 of the cuff 10 to communicate with the inlet of the air pump 22 and permits the outlet of the air pump 22 to the atmosphere. The selector valve 20 functions as cuff deflating means. The air pump 22 generates, at the inlet thereof, a vacuum whose pressure is lower than a pressure of the ambient atmosphere, i.e., atmospheric pressure. The air pump 22 functions as a vacuum or negative-pressure producing device.

The pressure signal SP is supplied to a low-pass filter 24 and a band-pass filter 28. The low-pass filter 24 permits only a static component of the pressure signal SP to pass therethrough, for thereby providing a cuff-pressure signal SK indicative of the air pressure of the cuff 10. The cuff-pressure signal SK is supplied to a central processing unit (CPU) 30 via an analog to digital (A/D) converter 26. The band-pass filter 28 permits only an oscillation component of the pressure signal SP to pass therethrough, for thereby providing a pulse wave signal SM indicative of a pressure oscillation produced in the cuff 10 in synchronism with heartbeat of the subject. The pulse wave signal SM is also supplied to the CPU 30 via the A/D converter 26.

The CPU 30 cooperates with a read only memory (ROM) 32, a random access memory (RAM) 34, and an interface 36 to constitute an arithmetic control device 37 which serves as blood pressure (BP) determining means. The CPU 30 processes input signals according to control programs pre-stored in the ROM 32 by utilizing a temporary-storage function of the RAM 34. The CPU 30 operates an output device 38 to display BP values of the subject, and controls the regulator valve 18, selector valve 20, and air pump 22.

The CPU 30 receives a first START signal MS from a START switch 40, a STOP signal MT from a STOP switch 42, and a second START signal CS from a START signal generator 44. A MANUAL/AUTO selector switch 46 is operable for selectively connecting one of the START switch 40 and the START signal generator 44 to the CPU 30. The START signal generator 44 includes a well-known flip-flop circuit, for example, and repeatedly generates the second START signal CS at a period of from about several minutes to about several tens of minutes pre-set through a period-setting device not shown.

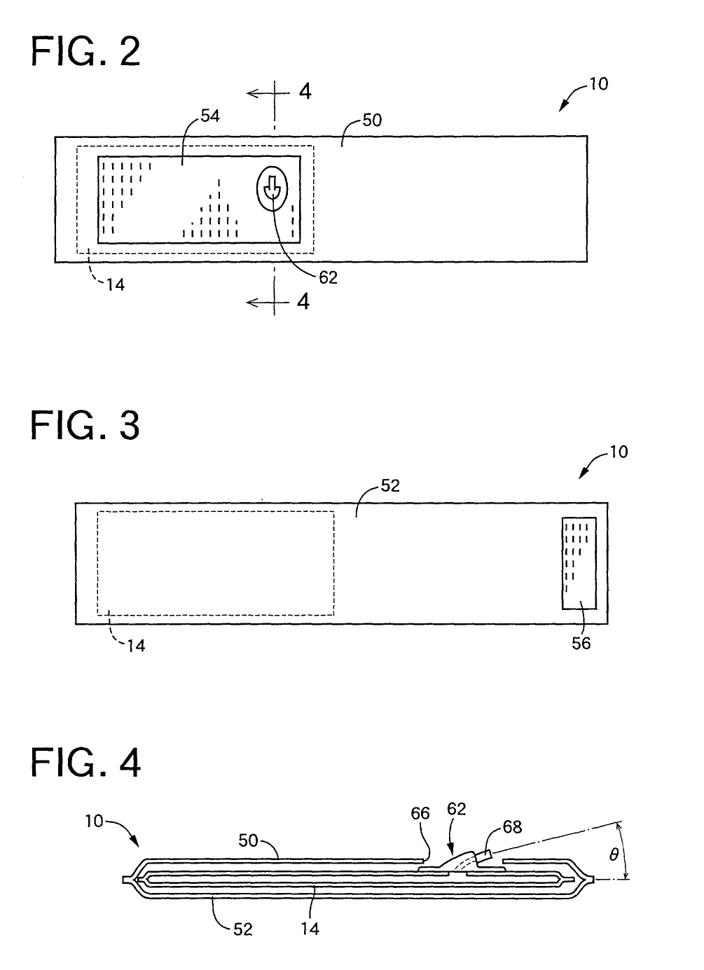

Referring next to FIGS. 2-4, there is shown the inflatable cuff 10 according to the present invention. FIGS. 2 and 3 show an outer surface and an inner surface of the cuff 10, respectively, while FIG. 4 shows a transverse cross-sectional view of the cuff 10 taken along line 4-4 of FIG. 2. As shown in FIGS. 2-4, the inflatable cuff 10 has an elongate bag-like configuration formed by an outer sheet member 50 and an inner sheet member 52 which are bonded together at their peripheral portions by various techniques such as sewing, bonding by using an adhesive agent, or high-frequency welding. Each of the outer and inner sheet members 50, 52 is a belt-like sheet member which is formed of a woven fabric of a synthetic resin, or a flexible synthetic-resin sheet such as a soft vinyl chloride sheet or a nylon sheet.

The inflatable bag 14 is accommodated in the elongate bag formed by the outer and inner sheet members 50, 52 described above, such that the inflatable bag 14 is located at one of two lengthwise opposite end portions of the cuff 10. The inflatable bag 14 has a rectangular configuration having a length dimension which is about half of that of the cuff 10.

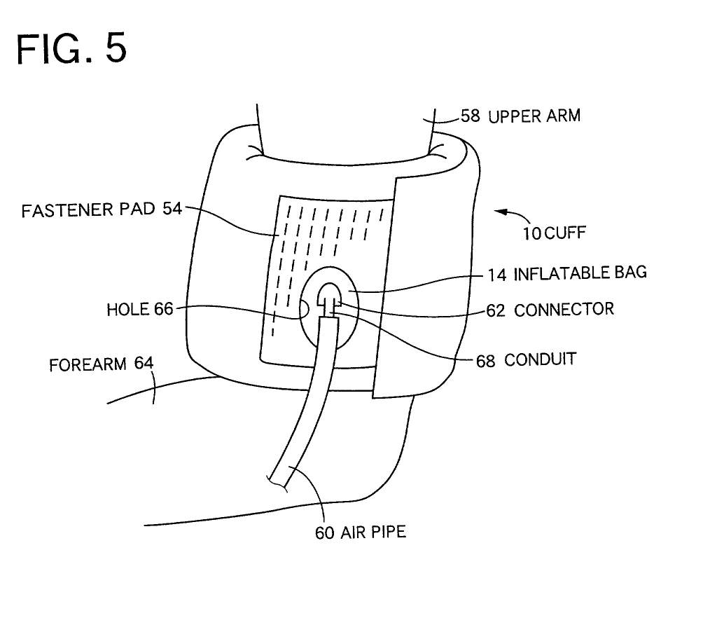

A pair of fastener pads 54, 56 are provided respectively on the outer surface of the cuff 10 provided by the outer sheet member 50 at one of the two lengthwise opposite end portions of the cuff 10, which end portion corresponds to the inflatable bag 14, and the inner surface of the cuff 10 provided by the inner sheet member 56 at the other of the two lengthwise opposite end portions which is remote from the inflatable bag 14. The fastener pads 54, 56 respectively have a multiplicity of looped fibers, and a multiplicity of hooks which are elastically deformable and which are formed of a synthetic resin. The multiplicity of looped fibers and the multiplicity of hooks are disengageably or detachable held in engagement with each other. As shown in FIG. 5, the cuff 10 is cylindrically wound around the body portion of the subject, e.g., an upper arm 58, such that one of the two lengthwise opposite end portions of the cuff 10 which is remote from the inflatable bag 14 is superposed on the other of the two lengthwise opposite end portions which corresponds to the inflatable bag 14, with the fastener pads 54, 56 being bonded to each other.

The inflatable bag 14 is formed of a relatively soft flexible member such as a vinyl chloride sheet, or a natural or a synthetic rubber sheet, and is air-tightly closed. The inflatable bag 14 is provided with a connector 62 for connecting the inflatable bag 14 to an air pipe 60 formed of an elastic material, through which a pressurized air is supplied from the air pump 22. The connector 62 is located at an intermediate position in a widthwise direction of the inflatable bag 14, such that the connector 62 is distant, by a predetermined distance, from a distal end of the cuff 10 in a widthwise direction thereof, which distal end of the cuff 10 is located on the side of a forearm 64 (FIG. 5) when the cuff 10 is wound around the upper arm 58. The position of the connector 62 corresponds to an intermediate portion of the cuff 10 in the widthwise direction thereof, and is determined so that the air pipe 60 connected to the connector 62 is not bent when the cuff 10 is wound around the upper arm 58 with the distal end of the cuff 10 being held in contact with the inner side of the forearm 64.

Like the air pipe 60, the connector 62 is a relatively soft elastic member in the form of a natural or a synthetic rubber sheet, for instance, and is fixed to the inflatable bag 14 by bonding using an adhesive agent, thermocompression bonding, or molding. The position of the inflatable bag 14 to which the connector 62 is attached is located, in the lengthwise direction of the cuff 10, at a position where the connector 62 is externally exposed in a state in which the cuff 10 is wound around the body portion of the subject such that the two lengthwise opposite end portions of the cuff 10 are superposed on each other with the pair of fastener pads 54, 56 being bonded to each other. The outer sheet member 50 of the cuff 10 is formed with an elliptical hole 66 at a position corresponding to the connector 62. The connector 62 is exposed to the outer surface of the cuff 10 through the hole 66.

The connector 62 includes a conduit 68 to which the air pipe 60 is connected for communication with the inflatable bag 14. The conduit 68 is formed integrally with the connector 62 such that the conduit 68 outwardly projects from the inflatable bag 14 such that the conduit 68 is inclined with respect to the inflatable bag 14 by an angle θ which falls in a range of from 20 degrees to 40 degrees with respect to the widthwise direction of the cuff 10 in a plane which is perpendicular to a plane of the cuff 10 and is parallel to the widthwise direction thereof. The conduit 68 is an elastic member similar to the connector 62.

In the present cuff wherein the connector 62, to which the air pipe 60 is connected for supplying a pressurized air to the inflatable bag 14, is attached to the inflatable bag 14 at a portion thereof which is distant, by a predetermined distance, from the distal end of the cuff 10 in the widthwise direction thereof, the air pipe 60 which is connected to the connector 62 is not bent even when the cuff 10 is wound around the body portion of the subject with the air pipe 60 contacting the inner side of the forearm 64, since the connector 62 is distant from the inner side of the forearm 58 by a distance enough to permit the air pipe 60 to extend in the direction parallel to the widthwise direction of the cuff 10 without being bent. Accordingly, the present arrangement is effective to prevent the skin of the subject from becoming sweaty and sodden due to an insufficient air flow through the air pipe experienced when the air pipe is bent without extending in the widthwise direction of the cuff.

In the present cuff 10 constructed as described above, the portion of the inflatable bag 14 to which the connector 62 is attached is located, in the lengthwise direction of the cuff 10, at a position where the connector 62 is externally exposed in a state in which the cuff 10 is wound around the body portion of the subject with the two lengthwise opposite end portions othe cuff 10 being superposed on each other. According to this arrangement, the connector 62 is not covered with one of the two lengthwise opposite end portions of the cuff 10 which is remote from the inflatable cuff 14 when the cuff 10 is cylindrically wound around the upper arm 58 of the subject with the above-indicated one of the two lengthwise opposite end portions of the cuff 10 which is remote from the inflatable bag 14 being superposed on the other of the two lengthwise opposite end portions. Accordingly, the air pipe 60 connected to the connector 62 is likely to be freely deformed, for thereby preventing the skin of the subject from becoming sweaty and sodden due to an insufficient air flow through the air pipe experienced when the air pipe is bent without extending in the widthwise direction of the cuff.

In the present cuff 10 wherein the connector 62 comprises the conduit 68 which is adapted to connect the inflatable bag 14 to the air pipe 60 and which outwardly projects from the inflatable bag 14 such that the conduit 68 is inclined with respect to the inflatable bag 14, the air pipe 60 is connected to the connector 62 via the conduit 68 such that the air pipe 60 is inclined with respect to the inflatable bag 14, for thereby preventing the air pipe from being bent.

In the present cuff 10, the conduit 68 is inclined with respect to the inflatable bag 14 by an angle θ which falls in a range of from 20 degrees to 40 degrees with respect to the widthwise direction of the cuff 10 in a plane which is perpendicular to a plane of the cuff 10 and is parallel to the widthwise direction thereof. According to this arrangement, the air pipe 60 is connected to the connector 62 via the conduit 68 such that the air pipe 60 is inclined with respect to the inflatable bag 14 by an angle θ which falls in a range of from 20 degrees to 40 degrees with respect to the widthwise direction of the cuff 10, for thereby effectively preventing the air pipe from being bent.

The connector 62 and the conduit 68 which is formed integrally with the connector 62 are formed of an elastic material and are elastically deformable. Even when the cuff 10 constructed according to the present embodiment is wound around a thin upper arm with the connector 62 being covered with one of the two lengthwise opposite end portions of the cuff 10 which is remote from the inflatable bag 14, the connector 62 is elastically deformed, facilitating winding of the cuff 10 around the upper arm, and permitting the air pipe 60 to extend in the widthwise direction of the cuff 10, so that air is permitted to easily flow through the air pipe 60.

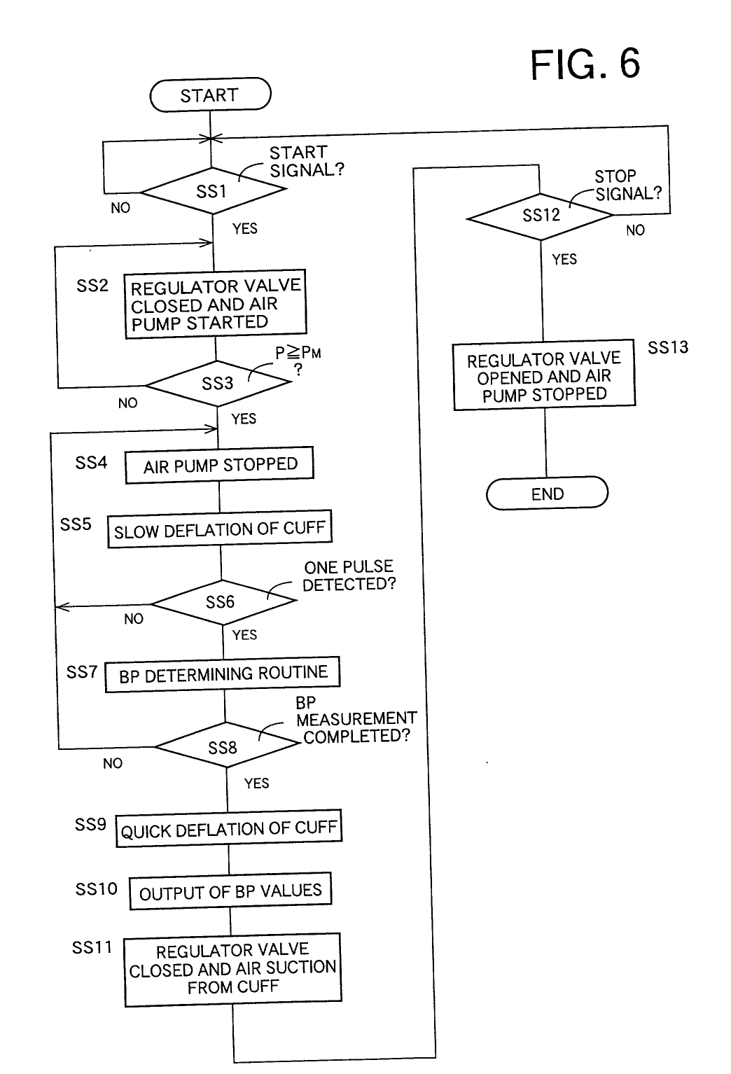

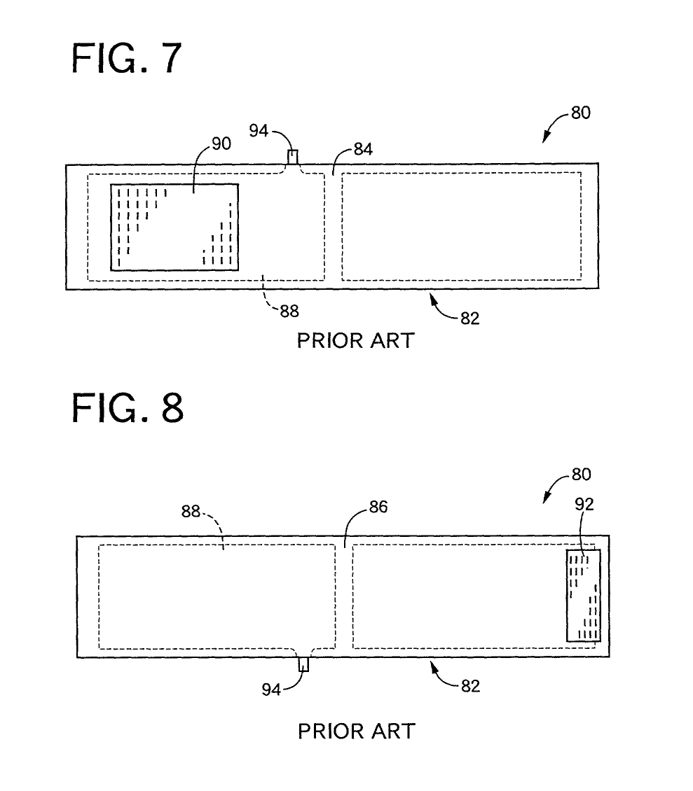

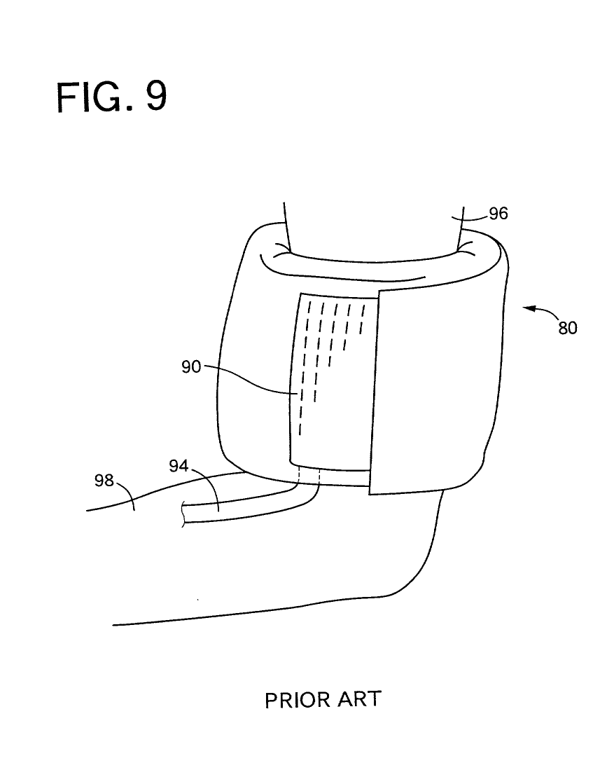

FIG. 6 shows a flow chart representing a control program which is carried out by the arithmetic control device 37. The control program is initiated with Step SS1 in which the CPU 30 judges whether the first or second START signal MS or CS is present at the CPU 30. If an affirmative judgment is made at step SS1, the control flow goes to Step SS2 to close the pressure regulator valve 18, place the selector valve 20 in the air-supplying position, and operate the air pump 22 so as to start inflation of the cuff 10, i.e., start increasing an air pressure P in the cuff 10. Step SS2 is followed by Step SS3 to judge whether the cuff pressure P has reached a predetermined reference value P_{M}. If a negative judgment is made in Step SS3, the CPU 30 repeats Steps SS2 and SS3. If an affirmative judgment is made in Step SS3, the control flow goes to Step SS4 to stop the air pump 22 and subsequently to Step SS5 to open the regulator valve 18 so as to start slow deflation of the cuff 10, i.e., start decreasing the cuff pressure P at a predetermined rate, e.g., about 2 to 3 mm/Hgsec.

Step SS5 is followed by Step SS6 to judge whether or not one pulse of the pulse wave signal SM corresponding to one heartbeat of the subject has been supplied form the cuff 10. If a negative judgment is made in Step SS6, Steps SS4 through SS6 are repeated. If an affirmative judgment is made in Step SS6, the control of the CPU 30 goes to Step SS7 to execute a blood pressure value determining routine. In this routine, the CPU 30 determines a cuff pressure P when the respective pulse amplitudes of the pulse wave signal SM have significantly largely increased, as a systolic blood pressure of the subject; determines a cuff pressure P when the pulse amplitudes have become maximum, as a mean blood pressure of the subject; and determines a cuff pressure P when the pulse amplitudes have significantly largely decreased, as a diastolic blood pressure of the subject. In the following Step SS8, the CPU 30 judges whether the blood pressure measurement in Step SS7 has been completed.

If a negative judgment is made in Step SS8, Steps SS4 through SS8 are repeated. If an affirmative judgment is made in Step SS8, the control flow goes to Step SS9 to open the regulator valve 18 so as to quickly deflate the cuff 10 in a predetermined time period. Step SS9 is followed by Step SS10 to operate the display device 38 to indicate the measured blood pressure values. Subsequently, Step SS11 is implemented to close the regulator valve 18, place the selector valve 20 in the air-discharging position, and operate the air pump 20 for a predetermined time period. Thus, the air remaining in the inflatable bag 14 is forcibly discharged therefrom to produce a vacuum or a negative pressure therein. Step SS11 is followed by Step SS12 to judge whether the STOP signal MT is present at the CPU 30. If a negative judgment is made in Step SS12, Steps SS1 through SS12 are repeatedly implemented. If an affirmative judgment is made in Step SS12, the control flow goes to Step SS13 to stop the air pump 22 and open the regulator valve 18.

The BP measuring apparatus 12 executes Step SS11 to produce a vacuum in the inflatable bag 14 of the cuff 10 during a non-BP measurement period following a BP measurement carried out in response to a manual operation of the START switch 40 or a periodic generation of a START signal from the signal generator 44. Since the inlet of the air pump 22 and the inflatable bag 14 are held in communication with each other through the selector valve 20, the air is discharged from the inflatable bag 14, and the cuff 10 is deflated, i.e., the thickness of the inflatable bag 14 is reduced during the non-BP measurement period. Accordingly, the area of contact of the cuff 10 with the skin of the subject's arm around which the cuff is wound, is minimized, so that air is permitted to more easily flow between the cuff and the patient's skin, thereby preventing the skin from becoming sweaty and sodden.

While the present invention has been described in its presently preferred embodiment, the invention may otherwise be embodied.

The cuff 10 of the illustrated embodiment includes the belt-like elongate bag and the inflatable bag 14 accommodated therein. The elongate bag of the cuff may be formed of an air-impermeable sheet member, and a portion of the elongate bag may define an inflatable bag.

The BP measuring apparatus 12 of the illustrated embodiment includes the vacuum producing device which produces a vacuum, so that the vacuum producing device sucks the air from the inflatable bag 14 and thereby forcibly deflates the inflatable bag 14, i.e., the thickness of the inflatable bag 14 is reduced, so as to minimize the area of contact of the cuff 10 which is wound around the body portion of the subject, and the skin of the body portion. It is to be understood that the cuff without such vacuum producing device assures the advantages described above with respect to the cuff 10 having the vacuum producing device.

In the cuff 10 of the illustrated embodiment, the air pipe 60 which is separate from the connector 62 is connected to the connector 62 for communication with the inflatable bag 14. The air pipe 60 and the connector 62 may be formed integrally with each other.

While the cuff 10 of the illustrated embodiment is a straight elongate member, the cuff 10 may be a curved elongate member as a whole.

It is to be understood that the present invention may be embodied with various other changes, modifications and improvements, which may occur to those skilled in the art, without departing from the spirit and scope of the invention defined in the following claims.

What is claimed is:

1. An inflatable cuff for being wound and inflated around a body portion of a living subject to press said body portion in measuring a blood pressure of the subject, the cuff comprising:
an inflatable bag which is located in a portion of the cuff in a lengthwise direction thereof; and
a connector which is attached to a portion of said inflatable bag that is distant, by a predetermined distance, from a distal end of the cuff in a widthwise direction thereof, and which is adapted to connect said inflatable bag to a gas-supply pipe to supply a pressurized gas from said pipe to said inflatable bag.

2. An inflatable cuff according to claim 1, wherein said portion of said inflatable bag to which said connector is attached is located, in the lengthwise direction of the cuff, at a position where said connector is externally exposed in a state in which the cuff is wound around said body portion of the subject with two lengthwise opposite end portions of the cuff being superposed on each other.

3. An inflatable cuff according to claim 1, wherein said connector comprises a conduit which is adapted to connect said inflatable bag to said gas-supply pipe and which outwardly projects from said inflatable bag such that said conduit is inclined with respect to said inflatable bag.

4. An inflatable cuff according to claim 3, wherein said conduit is inclined with respect to said inflatable bag by an angle which falls in a range of from 20 degrees to 40 degrees with respect to the widthwise direction of the cuff in a plane which is perpendicular to a plane of the cuff and is parallel to the widthwise direction thereof.

5. An inflatable bag according to claim 1, wherein said connector is formed of an elastic material and is elastically deformable.

6. An automatic blood pressure measuring apparatus, comprising:
an inflatable cuff according to claim 1;
a pressing-force changing device which changes a pressing force of the cuff applied to said body portion of the subject;
a determining device which determines a blood pressure of the subject based on a pulse wave produced in the cuff in synchronism with heartbeat of the subject while said pressing force of the cuff is changed by said pressing-force changing device;
a negative-pressure producing device which produces a negative pressure lower than an atmospheric pressure; and
a deflating device which communicates, after said determining means determines said blood pressure of the subject, said negative-pressure producing device with said inflatable bag of the cuff so that said negative-pressure producing device sucks the gas from said inflatable bag and thereby forcibly deflates said inflatable bag.
